(19) United States
(12) Patent Application Publication
(10) Pub. No.: US 2024/0269152 A1
(43) Pub. Date: Aug. 15, 2024

(54) NADES FORMULATIONS COMPRISING CANNABINOIDS

(71) Applicant: Clemson University, Clemson, SC (US)

(72) Inventors: Carlos D. Garcia, Clemson, SC (US); Lucas Ayres, Clemson, SC (US)

(21) Appl. No.: 18/421,523

(22) Filed: Jan. 24, 2024

Related U.S. Application Data
(60) Provisional application No. 63/441,929, filed on Jan. 30, 2023.

(51) Int. Cl. A61K 31/00; A61K 47/02; A61K 47/10; A61K 47/12; A61K 47/18

(52) U.S. Cl. CPC A61K 31/658; A61K 47/02; A61K 47/10; A61K 47/12; A61K 47/18; A61K 47/186

(57) ABSTRACT

A eutectic mixture or a pharmaceutical composition comprising the eutectic mixture wherein said eutectic mixture comprises a cannabinoid and at least one compound selected from an alkyl amine, an alcohol or a carboxylic acid and wherein the resulting mixture is a liquid at a given desired temperature where at least one of its components would, otherwise, be a solid unfit to be applied as a solvent.

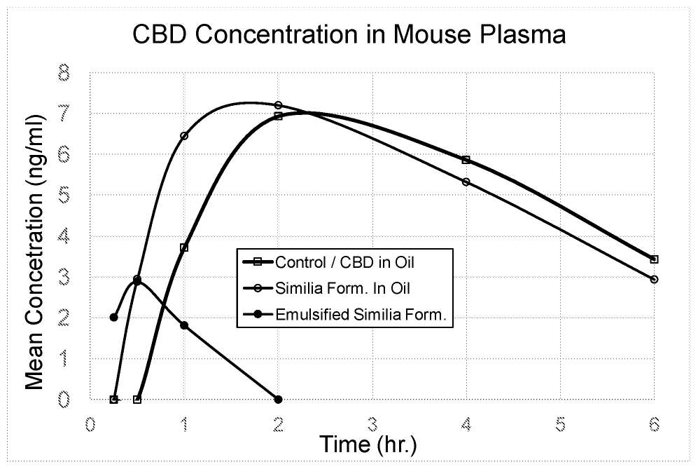

### CROSS-REFERENCE TO RELATED APPLICATIONS

The present application claims priority to pending U.S. Provisional Patent Application No. 63/441,929 filed Jan. 30, 2023 which is incorporated herein by reference.

### FIELD OF THE INVENTION

The present invention is related to formulations of novel natural deep eutectic solvents (NADES) comprising cannabinoids as a component of the NADES.

### BACKGROUND

Eutectic mixtures such as ionic liquids (IL), deep eutectic solvents (DES) and natural deep eutectic solvents (NADES) have been extensively studied as green alternative liquid media to traditional organic solvents. For the purposes of the present invention the primary focus will be on NADES without limit thereto.

DES and NADES are formed from specific ratios of multiple, typically two or three components leading to a system that is a liquid at a given desired temperature where at least one of its components would, otherwise, be a solid unfit to be applied as a solvent. The formation of eutectic solvents typically involves the formation of intrinsic interactions between the components, for example by moderate heating and stirring, rarely obtained by simply mixing the components. Of particular interest, without limit thereto, are those mixtures comprising components which when formed into a stable eutectic mixture, feature a melting point sufficiently low to be a liquid at ambient temperature, such as 20-30° C.

Eutectic mixtures have proven to be very beneficial for use in extracting various materials, for solubilizing various materials, and as excipients of several active pharmaceutical ingredients. The present invention is related to the extension of eutectics, and particularly NADES, that include pharmaceutically active ingredients (API) as a component of the NADES, wherein the melting point of the NADES incorporating the pharmaceutically active ingredient as one of its components is significantly low to be a liquid at ambient temperature and that typically feature better pharmacokinetic and/or pharmacodynamic profiles than the original pharmaceutically active ingredient.

### SUMMARY OF THE INVENTION

The present invention is related to the formation of a eutectic mixture comprising a pharmaceutically active ingredient as a component of the eutectic mixture wherein the eutectic mixture has a melting point lower than the pharmaceutically active ingredient, resulting in a liquid at ambient temperature.

A particular feature of the instant invention is the formation of a eutectic mixture comprising a cannabinoid as a component of the eutectic mixture.

A particular advantage of the instant invention is the ability to provide a formulation comprising the eutectic mixture with other components which are not a component of the eutectic mixture.

A particular advantage of the present invention is the ability to form a eutectic mixture of a cannabinoid wherein the cannabinoid forming the eutectic mixture is more readily delivered to mucosal membranes for transport through the mucosal membranes than as a cannabinoid alone or a cannabinoid dissolved in (or vehiculized by) other substances.

These, and other advantages, as will be realized, are provided in a eutectic mixture comprising a cannabinoid and a least one compound selected from the group consisting of an alkyl amine, an alcohol and a carboxylic acid wherein the eutectic mixture is stable and liquid at ambient temperature.

Yet another embodiment is provided in a pharmaceutical composition comprising a eutectic mixture. The eutectic mixture comprises a cannabinoid and a least one compound selected from the group consisting of an alkyl amine, an alcohol and a carboxylic acid. The eutectic mixture has a melting point low enough to remain liquid at ambient temperature.

### BRIEF DESCRIPTION OF FIGURE

FIGURE graphically illustrates the invention.

### DESCRIPTION

The present invention is related to a eutectic mixture comprising a cannabinoid as a component of the eutectic mixture. The eutectic mixture has a melting point which is low enough to remain liquid at ambient temperature. The eutectic mixture may be incorporated into a formulation comprising other components, such as solvents, which may or may not disrupt the intermolecular interactions of the eutectic mixture and therefore may or may not disrupt the eutectic mixture.

The inventive eutectic mixture comprises a cannabinoid and at least one of an alcohol or carboxylic acid and an alkyl amine and preferably a compound containing the both functionalities, such as alkynolamine. Formation of the eutectic mixture comprises mixing of the components, at a specific stoichiometric ratio, followed by introduction of energy. The amount of energy must be sufficient to disrupt the intermolecular interactions in the reactants, sometimes in solid forms, leading to new intermolecular interactions between the cannabinoid, alcohol or carboxylic acid and alkyl amine forming the eutectic mixture. Thermal processes are typically sufficient such as heating in a sealed vial, with agitation, at about 80° C. or more for an amount of time sufficient to promote the formation of the eutectic mixture. Other techniques can be employed to form the eutectic mixture such as ultrasonic mixing, microwave heating, solvent evaporation and the like.

It is particularly preferred that the eutectic mixture is stable for at least one week, wherein stable is defined as a eutectic mixture which does not separate into the components when left at ambient temperature for at least one week.

A cannabinoid is represented by General Formula:

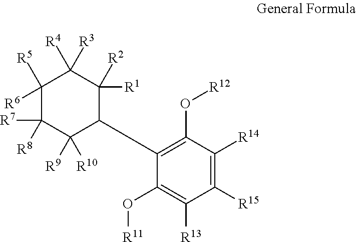

wherein:
R¹-R¹⁰ are independently selected from H or an alkyl of 1-10 carbons; alkenyl of up to 10 carbons; or groups on adjacent carbons may be taken together to form an alkene; R¹, R⁵, R⁶, R⁷, R⁸ and R⁹ are preferably H; R² and R³ are preferably taken together to form an alkene; R⁴ is preferably —CH₃; R¹⁰ is preferably —C(═CH₂)CH₃; R¹¹ and R¹² are independently H or R¹¹ and R¹⁰ may be taken together to represent —C(R¹⁹)₂—; R¹³-R¹⁵ independently represent H or an alkyl of 1-8 carbons; R¹³ and R¹⁴ are preferably H; R¹⁵ is preferably an alkyl of 1-8 carbons and more preferably —(CH₂)₄CH₃; and each R¹⁹ independently represents H or an alkyl of 1-5 carbons; each R¹⁹ is preferably —CH₃.

A particularly preferred emulsifier of the General Formula is represented by Formula I:

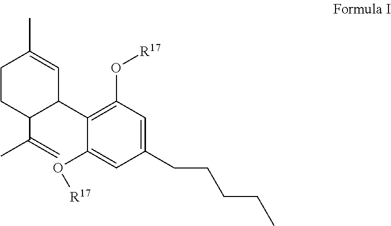

In a particularly preferred embodiment of Formula I, each R¹⁷ is independently selected from H, C(O)C₆H₄OH, —C(O)CH═CHC₆H₅, —C(O)(CH₂)₇CH₃, or —C(O)(CH₂)₉CH₃ and preferably both R¹⁷ groups are the same. A particularly preferred cannabinoid is cannabidiol wherein each R¹⁷ of Formula I is hydrogen.

In another particularly preferred embodiment of the General Formula R¹⁰ and R¹¹ are taken together as —C(CH₃)₂— as represented by Formula II:

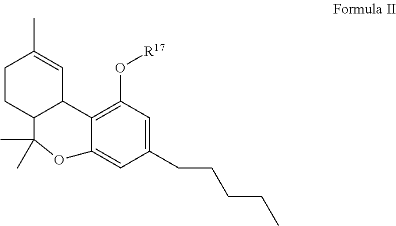

In a particularly preferred embodiment of Formula II, each R¹⁷ is independently selected from H, C(O)C₆H₄OH, —C(O)CH═CHC₆H₅, —C(O)(CH₂)₇CH₃, or —C(O)(CH₂)₉CH₃. A particularly preferred cannabinoid is tetrahydrocannabinol wherein R¹⁷ of Formula II is hydrogen.

A particularly preferred NADES formulation comprises a cannabinoid either synthetic or obtained from cannabis. Particularly preferred cannabinoids are selected from the group consisting of cannabidiol (CBD), tetrahydrocannabinol (THC), cannabichromene (CBC), cannabichromevarin (CBCV), cannabidiphorol (CBDP), cannabidivarinic acid (CBDVA), cannabielsoin (CBEA), cannabigerolic acid (CBCA), cannabigerolic acid monomethyl ether (CBGAM), cannabigerovarinic acid (CBGVA), cannabicyclolic acid (CBLA), cannabinolic acid (CBNA), cannabicitranic acid (CBTA), cannabivarinic acid (CBVA), tetrahydrocannabinolic acid (THCA), tetrahydrocannabiorcolic acid (THCCA), tetrahydrocannabiphorolic acid (THCPA), tetrahydrocannabinolic acid (THCA), tetrahydrocannabivarinic acid (THCVA), cannadibiphorol (CBDP), cannabidivarin (CBDV), cannabielsoin (CBE), cannabigerol (CBG), cannabigerol monomethylether (CBGM), cannabigerovarin (CBG), cannabigerol monomethylether (CBGM), cannabigerovarin (CBGV), cannabicyclol (CBL), cannabinol (CBN), cannabicitran (CBTC), cannabivarin (CBV), tetrahydrocannabiorcol (THCC), tetrahydrocannabiphorol (THCP) or tetrahydrocannabivarin (TNCV). Other products from cannabis may be used in to form a NADES.

Particularly preferred alcohols or carboxylic acids include mono-alcohols such as methanol, propane-1-ol, hexafluoroisopropanolmenthol, tetradecane-1-ol, 1-tetradecanol, borneol or vanillin; polyols such as ethylene glycol, glycerol, ethane-1,2-diol, butane-1,2-diol, butane-1,3-diol, 1,4 butanediol, butane-1,2,3,4-tetrol, 1,3-propanediol, 1,6-hexanediol, pentinol, hexitol or ribose particularly D-ribose; sugars such as glucose, galactose, lactose, maltose particularly alpha maltose and alpha-D-glucopyranoside; carboxylic acids such as tetradecanoic acid, 2-aminopentanedioic acid, lauric acid, nonanoic acid, formic acid, dodecanoic acid, adipic acid, stearic acid, phthalic acid, undec-10-enoic acid, undecanoic acid, 1,3,4,5,6-pentahydroxyhexan-2-one, glutamic acid, octanoic acid, palmitic acid, hexanoic acid, trimesic acid and combinations thereof such as sorbose or laurinol.

Ketones such as camphor or nonanol are also suitable for use in a eutectic.

A preferred amine is urea. The alkyl amine is preferably an ethyl amine, more preferably an alkynol amine most preferably comprising an ethanol amine (HOCH₂CH₂N—) group. A particularly preferred alkyl amine is 1-(diethylamino)ethanol hydrochloride or threonine and particularly DL-threonine.

Particularly preferred alkyl amines include azanium halides and particularly alkyl azanium halides such as ethylazanium chloride or tetraethylazanium chloride, choline halide and particularly choline chloride or choline flouride, ethyl-(2-hydroxyethyl)-dimethylazanium chloride, diethyl-(2-hydroxyethyl)-azanium chloride, benzyl(triethyl)azanium chloride, benzyl(trimethyl)azanium chloride and tetrabutylazanium chloride; 2-[bis(2-hydroxyethyl)amino]ethanol, 2-(2-hydroxyethylamino)ethanol, 2-aminoethanol, tetraethylamine chloride, 1,3 dimethyl urea, 1,1-dimethyl urea, trimethyl glycine (betaine), carnitine, ethyl(2-hydroxyethyl)dimethylammonium chloride, diethanolamine and ethanol amine.

Additional components of the eutectic mixture include bases such as ammonium thiocyanate, dichlorozinc, boric acid, sodium acetate, eucalyptol and eucalyptus oil.

The eutectic is in a stoichiometric relationship. Therefore, the molar ratio of cannabinoid to each other component of a binary eutectic are typically at least 1:10 to no more than 10:1 and more preferably at least 1:5 to no more than 5:1. Mixtures containing more than two components (such as ternary, quaternary, etc) are also possible.

Exemplary embodiments are selected from the group consisting of: 0.9-1.1 part ethylazanium chloride, 0.9-1.1 part tetradecanoic acid and 4.5-5.5 parts CBD; 0.9-1.1 part propan-1-ol, 2.7-3.3 parts choline chloride and 0.9-1.1 part CBD; 0.9-1.1 part choline chloride, 0.9-1.1 part ethyl-(2-hydroxyethyl)-dimethylazanium chloride, 2.7-3.3 parts menthol and 4.5-5.5 parts CBD; 0.9-1.1 part choline chloride, 2.7-3.3 parts 2-aminopentanedioic acid, 2.7-3.3 parts CBD and 1.8-2.2 parts ethylazanium chloride; 0.9-1.1 part 2-[bis(2-hydroxyethyl)amino]ethanol, 0.9-1.1 part water and 4.5-5.5 parts CBD; 0.9-1.1 part 2-(2-hydroxyethylamino)ethanol, 0.9-1.1 part tetradecan-1-ol and 4.5-5.5 parts CBD; 0.9-1.1 part choline chloride, 0.9-1.1 part undec-10-enoic acid, 0.9-1.1 part 2-[bis(2-hydroxyethyl)amino]ethanol and 4.5-5.5 parts CBD; 1.8-2.2 parts glycerol, 0.9-1.1 part dichlorozinc and 0.9-1.1 part CBD; 0.9-1.1 part choline chloride, 3.6-4.4 parts CBD and 4.5-5.5 parts urea; 2.7-3.3 parts butane-1,2-diol, 0.9-1.1 part ethylene glycol and 0.9-1.1 part CBD; 1.8-2.2 parts choline chloride, 2.7-3.3 parts benzyl(triethyl)azanium chloride, 0.9-1.1 part CBD, 0.9-1.1 part 1,6-hexanediol and 0.9-1.1 part lauric acid; 0.9-1.1 part (CH₃CH₂)₄N⁺Cl⁻, 0.9-1.1 part propan-1-ol, 0.9-1.1 part CBD and 0.9-1.1 part CH₃(CH₂)₇COOH; 0.9-1.1 part choline chloride, 2.7-3.3 parts CBD and 4.5-5.5 parts 1,3-dimethylurea; 0.9-1.1 part choline chloride, 1.8-2.2 parts CBD, 2.7-3.3 parts formic acid and 4.5-5.5 parts betaine; 0.9-1.1 part choline chloride, 3.6-4.4 parts ethanolamine, 0.9-1.1 part CBD and 1.8-2.2 parts glycerine; 0.9-1.1 part choline chloride, 4.5-5.5 parts dodecanoic acid, 3.6-4.4 parts adipic acid and 0.9-1.1 part CBD; 0.9-1.1 part ethyl-(2-hydroxyethyl)-dimethylazanium chloride, 1.8-2.2 parts CBD and 1.8-2.2 parts ethylazanium chloride; 1.8-2.2 parts ethanolamine, 0.9-1.1 part propan-1-ol and 1.8-2.2 parts CBD; 1.8-2.2 parts 1,4 butanediol, 1.8-2.2 parts 1,3-propanediol and 3.6-4.4 parts CBD; 0.9-1.1 part ethyl-(2-hydroxyethyl)-dimethylazanium chloride, 2.7-3.3 parts dichlorozinc, 1.8-2.2 parts CBD, 1.8-2.2 parts pentitol and 0.9-1.1 part 2-(2-hydroxyethylamino)ethanol; 0.9-1.1 part ethane-1,2-diol, 2.7-3.3 parts ethylazanium chloride and 1.8-2.2 parts CBD; 4.5-5.5 parts choline chloride, 4.5-5.5 parts 1-tetradecanol and 4.5-5.5 parts CBD; 0.9-1.1 part choline chloride, 1.8-2.2 parts tetrabutylazanium chloride, 2.7-3.3 parts sorbose and 2.7-3.3 parts CBD; 0.9-1.1 part borneol, 0.9-1.1 part CBD, 0.9-1.1 part 1,3-propanediol and 3.6-4.4 parts stearic acid; 0.9-1.1 part formic acid, 0.9-1.1 part CBD and 4.5-5.5 parts 1-tetradecanol; 4.5-5.5 parts CBD, 0.9-1.1 part menthol and 0.9-1.1 part lauric acid; 1.8-2.2 parts CBD, 0.9-1.1 part menthol; 2.7-3.3 parts CBD, 1.8-2.2 parts menthol and 0.9-1.1 part lauric acid; 2.7-3.3 parts CBD, 0.9-1.1 part menthol and 0.9-1.1 part glycol; 2.7-3.3 parts CBD, 0.9-1.1 part undecanoic acid and 0.9-1.1 part eucalyptol.

Exemplary embodiments are listed in Table 1.

TABLE 1

| # | Components |
|---|---|
| 1 | 1 part ethylazanium chloride, 1 part tetradecanoic acid and 5 parts CBD |
| 2 | 1 part propan-1-ol, 3 parts choline chloride and 1 part CBD |
| 3 | 1 part choline chloride, 1 part ethyl-(2-hydroxyethyl)-dimethylazanium chloride, |
| | 3 parts menthol and 5 parts CBD |
| 4 | 1 part choline chloride, 3 parts 2-aminopentanedioic acid, 3 parts CBD and 2 |
| | parts ethylazanium chloride |
| 5 | 1 part 2-[bis(2-hydroxyethyl)amino]ethanol, 1 part water and 5 parts CBD |
| 6 | 1 part 2-(2-hydroxyethylamino)ethanol, 1 part tetradecan-1-ol and 5 parts CBD |
| 7 | 1 part choline chloride, 1 part undec-10-enoic acid, 1 part 2-[bis(2- |
| | hydroxyethyl)amino]ethanol and 5 parts CBD |
| 8 | 2 parts glycerol, 1 part dichlorozinc and 1 part CBD |
| 9 | 1 part choline chloride, 4 parts CBD and 5 parts urea |
| 10 | 3 parts butane-1,2-diol, 1 part ethylene glycol and 1 part CBD |
| 11 | 2 parts choline chloride, 3 parts benzyl(triethyl)azanium chloride, 1 part CBD, 1 |
| | part 1,6-hexanediol and 1 part lauric acid |
| 12 | 1 part (CH₃CH₂)₄N⁺Cl⁻, 1 part propan-1-ol, 1 part CBD and 1 part |
| | CH₃(CH₂)₇COOH |
| 13 | 1 part choline chloride, 3 parts CBD and 5 parts 1,3-dimethylurea |
| 14 | 1 part choline chloride, 2 parts CBD, 3 parts formic acid and 5 parts betaine |
| 15 | 1 part choline chloride, 4 parts ethanolamine, 1 part CBD and 2 parts glycerine |
| 16 | 1 part choline chloride, 5 parts dodecanoic acid, 4 parts adipic acid and 1 part |
| | CBD |
| 17 | 1 part ethyl-(2-hydroxyethyl)-dimethylazanium chloride, 2 parts CBD and 2 parts |
| | ethylazanium chloride. |
| 18 | 2 parts ethanolamine, 1 part propan-1-ol and 2 parts CBD |
| 19 | 2 parts 1,4 butanediol, 2 parts 1,3-propanediol and 4 parts CBD |
| 20 | 1 part ethyl-(2-hydroxyethyl)-dimethylazanium chloride, 3 parts dichlorozinc, 2 |
| | parts CBD, 2 parts pentitol and 1 part 2-(2-hydroxyethylamino)ethanol |
| 21 | 1 part ethane-1,2-diol, 3 parts ethylazanium chloride and 2 parts CBD |
| 22 | 5 parts choline chloride, 5 parts 1-tetradecanol and 5 parts CBD |
| 23 | 1 part choline chloride, 2 parts tetrabutylazanium chloride, 3 parts sorbose and |
| | 3 parts CBD |
| 24 | 1 part borneol, 1 part CBD, 1 part 1,3-propanediol and 4 parts stearic acid |
| 25 | 1 part formic acid, 1 part CBD and 5 parts 1-tetradecanol |
| 26 | 5 parts CBD, 1 part menthol and 1 part lauric acid |
| 27 | 2 parts CBD, 1 part menthol |
| 28 | 3 parts CBD, 2 parts menthol and 1 part lauric acid |
| 29 | 3 parts CBD, 1 part menthol and 1 part glycol |
| 30 | 3 parts CBD, 1 part undecanoic acid and 1 part eucalyptol |

In Table 1 “part” refers to the molar ratio of the component wherein the molar ratio is +10%. By way of example, a composition listing 1 part A, 2 parts B and 5 parts C indicates 0.9-1.1 part A, 1.8-2.2 parts B and 4.5-5.5 parts C.

It is particularly preferred to utilize the eutectic mixture in a pharmaceutical formulation comprising 1 to 99 wt % carriers and additional components. Particularly preferred carriers include solvents, particularly oils and most particularly natural oils. Additional components comprise active ingredients such as cough suppressants, aromatics, analgesics and the like for multi-purpose pharmaceuticals. Eucalyptol and eucalyptus oil are particularly suitable components of the pharmaceutical formulation.

### Examples

### Pharmacokinetic Test

Uptake of CBD was determined by analyzing the concentration of CBD in the blood of mice as a function of time. Three mice were studied for each sample. Samples were administered by oral ingestion with blood drawn and tested after 15 minutes, 30 minutes, 1 hour, 2 hours, 4 hours and 6 hours after administration. Sampling was adjusted to insure the amount of CBD was consistent for each mouse and to be consistent with human dosage at 50 mg/Kg. Due to the small sample size each sample was diluted with coconut oil which is consistent with teachings in the art.

The samples were separated using liquid chromatography in a Waters Xbridge C18, 2.1×30 mm, 3.5 μm column, at 45° C. Mobile phase A comprised about 94.9 v % water, about 5 v % acetonitrile and about 0.1 v % formic acid. Mobile phase B comprised about 49.9 v % methanol, about 50 v % acetonitrile and about 0.1 v % formic acid. The CBD was detected by mass spectroscopy using a API-400 mass spectrometer with electrospray as the ionization technique at a source temperature of 550° C., in positive ion mode.

A first control sample was prepared by dissolving CBD in coconut oil. A second control sample was prepared utilizing nanoemulsions produced by high-intensity ultrasounds referred to as an Emulsified Similia Form. The formation of nanoemulsions has been proposed as a method of improving bioavailability of cannabinoids.

The inventive formulation, referred to as Similia Form, was prepared by 2 parts CBD with 1 part menthol, per mole, followed by mild heating and ultrasonication, until the eutectic was formed.

The results are reported in the FIGURE wherein the first control indicated observable CBD at 1 hour versus the inventive formulation wherein observable CBD was detected at 30 minutes. The rate of absorption with the inventive formulation was about twice the rate of absorption of the first control with a cumulative bioavailability of approximately 3× at 1 hour and 1.5× for hours 1-2. The nanoemulsion was inferior to the inventive example and the first control.

The invention has been described with reference to preferred embodiments without limit thereto. One of skill in the art would realize additional embodiments which are described and set forth in the claims appended hereto.

1. A eutectic mixture comprising:
a cannabinoid and a least one compound selected from the group consisting of an alkyl amine, an alcohol and a carboxylic acid.
wherein said eutectic mixture has a melting point low enough to remain liquid at ambient temperature.

2. The eutectic mixture of claim 1 wherein said cannabinoid is defined by General Formula: 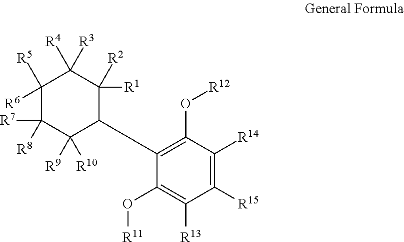
wherein:
R¹-R¹⁰ are independently selected from H or an alkyl of 1-10 carbons; alkenyl of up to 10 carbons; or groups on adjacent carbons may be taken together to form an alkene;
R¹¹ and R¹² are independently H or R¹¹ and R¹⁰ may be taken together to represent —C(R¹⁹)₂—;
R¹³-R¹⁵ independently represent H or an alkyl of 1-8 carbons; and
each R¹⁹ independently represents H or an alkyl of 1-5 carbons.

3. The eutectic mixture of claim 2 wherein each of R¹, R⁵, R⁶, R⁷, R⁸ and R⁹ is H.

4. The eutectic mixture of claim 2 wherein R² and R³ are taken together to form an alkene.

5. The eutectic mixture of claim 2 wherein R⁴ is —CH₃.

6. The eutectic mixture of claim 2 wherein R¹⁰ is —C(═CH₂)CH₃;

7. The eutectic mixture of claim 2 wherein R¹³ and R¹⁴ are H.

8. The eutectic mixture of claim 2 wherein R¹⁵ is an alkyl of 1-8 carbons.

9. The eutectic mixture of claim 8 wherein R¹⁵ is —(CH₂)₄CH₃.

10. The eutectic mixture of claim 2 wherein each R¹⁹ is —CH₃.

11. The eutectic mixture of claim 2 wherein said cannabinoid is defined by Formula I: 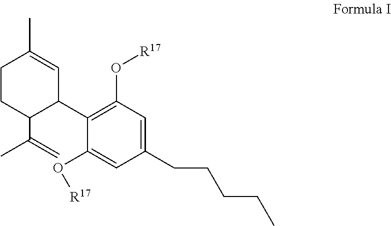
wherein each R¹⁷ is independently selected from H, C(O)C₆H₄OH, —C(O)CH═CHC₆H₅, —C(O)(CH₂)₇CH₃, or —C(O)(CH₂)₉CH₃.

12. The eutectic mixture of claim 11 wherein both R¹⁷ groups are the same.

13. The eutectic mixture of claim 12 wherein both R¹⁷ groups are hydrogen.

14. The eutectic mixture of claim 2 wherein said cannabinoid is defined by Formula II: 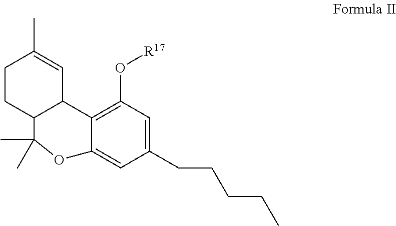
wherein R¹⁷ is independently selected from H, C(O)C₆H₄OH, —C(O)CH═CHC₆H₅, —C(O)(CH₂)₇CH₃, or —C(O)(CH₂)₉CH₃.

15. The eutectic mixture of claim 14 wherein R¹⁷ is hydrogen.

16. The eutectic mixture of claim 1 wherein said cannabinoid is selected from the group consisting of cannabidiol, tetrahydrocannabinol, cannabichromene, cannabichromevarin, cannabidiphorol, cannabidivarinic acid, cannabielsoin, cannabigerolic acid, cannabigerolic acid monomethyl ether, cannabigerovarinic acid, cannabicyclolic acid, cannabinolic acid, cannabicitranic acid, cannabivarinic acid, tetrahydrocannabinolic acid, tetrahydrocannabiorcolic acid, tetrahydrocannabiphorolic acid, tetrahydrocannabinolic acid, tetrahydrocannabivarinic acid, cannadibiphorol, cannabidivarin, cannabielsoin, cannabigerol, cannabigerol monomethylether, cannabigerovarin, cannabigerol monomethylether, cannabigerovarin, cannabicyclol, cannabinol, cannabicitran, cannabivarin, tetrahydrocannabiorcol, tetrahydrocannabiphorol and tetrahydrocannabivarin.

17. The eutectic mixture of claim 16 wherein said cannabinoid is selected from the group consisting of cannabidiol and tetrahydrocannabinol.

18. The eutectic mixture of claim 17 wherein said cannabinoid is cannabidiol tetrahydrocannabinol.

19. The eutectic mixture of claim 1 comprising a cannabinoid and an alkyl amine.

20. The eutectic mixture of claim 19 wherein alkyl amine is an alkynol amine.

21. The eutectic mixture of claim 19 wherein alkyl amine is selected from the group consisting of alkyl azanium halides, choline halide, ethyl-(2-hydroxyethyl)-dimethylazanium chloride, diethyl-(2-hydroxyethyl)-azanium chloride, benzyl(triethyl)azanium chloride, benzyl(trimethyl)azanium chloride and tetrabutylazanium chloride; 2-[bis(2-hydroxyethyl)amino]ethanol, 2-(2-hydroxyethylamino)ethanol, 2-aminoethanol, tetraethylamine chloride, 1,3 dimethyl urea, 1,1-dimethyl urea, trimethyl glycine (betaine), carnitine, ethyl(2-hydroxyethyl)dimethylammonium chloride, diethanolamine and ethanol amine.

22. The eutectic mixture of claim 21 wherein azanium halides is selected from the group consisting of ethylazanium chloride, choline chloride, choline fluoride, ethyl-(2-hydroxyethyl)-dimethylazanium chloride, benzyl(triethyl)azanium chloride and tetrabutylazanium chloride.

23. The eutectic mixture of claim 1 comprising a cannabinoid and an alcohol.

24. The eutectic mixture of claim 23 wherein said alcohol is selected from a mono-alcohols and a polyol.

25. The eutectic mixture of claim 24 wherein said alcohol is selected from the group consisting of methanol, propane-1-ol, hexafluoroisopropanolmenthol, tetradecane-1-ol, 1-tetradecanol, borneol, vanillin, ethylene glycol, glycerol, ethane-1,2-diol, butane-1,2-diol, butane-1,3-diol, 1,4 butanediol, butane-1,2,3,4-tetrol, 1,3-propanediol, 1,6-hexanediol, pentinol, hexitol, ribose, glucose, galactose, lactose, maltose and alpha-D-glucopyranoside.

26. The eutectic mixture of claim 1 comprising a cannabinoid and a carboxylic acid.

27. The eutectic mixture of claim 26 wherein said carboxylic acid is selected from the group consisting of tetradecanoic acid, 2-aminopentanedioic acid, lauric acid, nonanoic acid, formic acid, dodecanoic acid, adipic acid, stearic acid, phthalic acid, undec-10-enoic acid, undecanoic acid, 1,3,4,5,6-pentahydroxyhexan-2-one, glutamic acid, octanoic acid, palmitic acid, hexanoic acid and trimesic acid.

28. The eutectic mixture of claim 1 selected from the group consisting of: 0.9-1.1 part ethylazanium chloride, 0.9-1.1 part tetradecanoic acid and 4.5-5.5 parts CBD; 0.9-1.1 part propan-1-ol, 2.7-3.3 parts choline chloride and 0.9-1.1 part CBD; 0.9-1.1 part choline chloride, 0.9-1.1 part ethyl-(2-hydroxyethyl)-dimethylazanium chloride, 2.7-3.3 parts menthol and 4.5-5.5 parts CBD; 0.9-1.1 part choline chloride, 2.7-3.3 parts 2-aminopentanedioic acid, 2.7-3.3 parts CBD and 1.8-2.2 parts ethylazanium chloride; 0.9-1.1 part 2-[bis(2-hydroxyethyl)amino]ethanol, 0.9-1.1 part water and 4.5-5.5 parts CBD; 0.9-1.1 part 2-(2-hydroxyethylamino)ethanol, 0.9-1.1 part tetradecan-1-ol and 4.5-5.5 parts CBD; 0.9-1.1 part choline chloride, 0.9-1.1 part undec-10-enoic acid, 0.9-1.1 part 2-[bis(2-hydroxyethyl)amino]ethanol and 4.5-5.5 parts CBD; 1.8-2.2 parts glycerol, 0.9-1.1 part dichlorozinc and 0.9-1.1 part CBD; 0.9-1.1 part choline chloride, 3.6-4.4 parts CBD and 4.5-5.5 parts urea; 2.7-3.3 parts butane-1,2-diol, 0.9-1.1 part ethylene glycol and 0.9-1.1 part CBD; 1.8-2.2 parts choline chloride, 2.7-3.3 parts benzyl(triethyl)azanium chloride, 0.9-1.1 part CBD, 0.9-1.1 part 1,6-hexanediol and 0.9-1.1 part lauric acid; 0.9-1.1 part (CH₃CH₂)₄N⁺Cl⁻, 0.9-1.1 part propan-1-ol, 0.9-1.1 part CBD and 0.9-1.1 part CH₃(CH₂)₇COOH; 0.9-1.1 part choline chloride, 2.7-3.3 parts CBD and 4.5-5.5 parts 1,3-dimethylurea; 0.9-1.1 part choline chloride, 1.8-2.2 parts CBD, 2.7-3.3 parts formic acid and 4.5-5.5 parts betaine; 0.9-1.1 part choline chloride, 3.6-4.4 parts ethanolamine, 0.9-1.1 part CBD and 1.8-2.2 parts glycerine; 0.9-1.1 part choline chloride, 4.5-5.5 parts dodecanoic acid, 3.6-4.4 parts adipic acid and 0.9-1.1 part CBD; 0.9-1.1 part ethyl-(2-hydroxyethyl)-dimethylazanium chloride, 1.8-2.2 parts CBD and 1.8-2.2 parts ethylazanium chloride; 1.8-2.2 parts ethanolamine, 0.9-1.1 part propan-1-ol and 1.8-2.2 parts CBD; 1.8-2.2 parts 1,4 butanediol, 1.8-2.2 parts 1,3-propanediol and 3.6-4.4 parts CBD; 0.9-1.1 part ethyl-(2-hydroxyethyl)-dimethylazanium chloride, 2.7-3.3 parts dichlorozinc, 1.8-2.2 parts CBD, 1.8-2.2 parts pentitol and 0.9-1.1 part 2-(2-hydroxyethylamino)ethanol; 0.9-1.1 part ethane-1,2-diol, 2.7-3.3 parts ethylazanium chloride and 1.8-2.2 parts CBD; 4.5-5.5 parts choline chloride, 4.5-5.5 parts 1-tetradecanol and 4.5-5.5 parts CBD; 0.9-1.1 part choline chloride, 1.8-2.2 parts tetrabutylazanium chloride, 2.7-3.3 parts sorbose and 2.7-3.3 parts CBD; 0.9-1.1 part borneol, 0.9-1.1 part CBD, 0.9-1.1 part 1,3-propanediol and 3.6-4.4 parts stearic acid; 0.9-1.1 part formic acid, 0.9-1.1 part CBD and 4.5-5.5 parts 1-tetradecanol; 4.5-5.5 parts CBD, 0.9-1.1 part menthol and 0.9-1.1 part lauric acid; 1.8-2.2 parts CBD, 0.9-1.1 part menthol; 2.7-3.3 parts CBD, 1.8-2.2 parts menthol and 0.9-1.1 part lauric acid; 2.7-3.3 parts CBD, 0.9-1.1 part menthol and 0.9-1.1 part glycol; 2.7-3.3 parts CBD, 0.9-1.1 part undecanoic acid and 0.9-1.1 part eucalyptol.

29. The eutectic mixture of claim 28 selected from the group consisting of: selected from the group consisting of: 1 part ethylazanium chloride, 1 part tetradecanoic acid and 5 parts CBD; 1 part propan-1-ol, 3 parts choline chloride and 1 part CBD; 1 part choline chloride, 1 part ethyl-(2-hydroxyethyl)-dimethylazanium chloride, 3 parts menthol and 5 parts CBD; 1 part choline chloride, 3 parts 2-aminopentanedioic acid, 3 parts CBD and 2 parts ethylazanium chloride; 1 part 2-[bis(2-hydroxyethyl)amino]ethanol, 1 part water and 5 parts CBD; 1 part 2-(2-hydroxyethylamino)ethanol, 1 part tetradecan-1-ol and 5 parts CBD; 1 part choline chloride, 1 part undec-10-enoic acid, 1 part 2-[bis(2-hydroxyethyl)amino]ethanol and 5 parts CBD; 2 parts glycerol, 1 part dichlorozinc and 1 part CBD; 1 part choline chloride, 4 parts CBD and 5 parts urea; 3 parts butane-1,2-diol, 1 part ethylene glycol and 1 part CBD;_2 parts choline chloride, 3 parts benzyl(triethyl)azanium chloride, 1 part CBD, 1 part 1,6-hexanediol and 1 part lauric acid; 1 part (CH₃CH₂)₄N⁺Cl⁻, 1 part propan-1-ol, 1 part CBD and 1 part CH₃(CH₂)₇COOH; 1 part choline chloride, 3 parts CBD and 5 parts 1,3-dimethylurea; 1 part choline chloride, 2 parts CBD, 3 parts formic acid and 5 parts betaine; 1 part choline chloride, 4 parts ethanolamine, 1 part CBD and 2 parts glycerine; 1 part choline chloride, 5 parts dodecanoic acid, 4 parts adipic acid and 1 part CBD; 1 part ethyl-(2-hydroxyethyl)-dimethylazanium chloride, 2 parts CBD and 2 parts ethylazanium chloride; 2 parts ethanolamine, 1 part propan-1-ol and 2 parts CBD; 2 parts 1,4 butanediol, 2 parts 1,3-propanediol and 4 parts CBD; 1 part ethyl-(2-hydroxyethyl)-dimethylazanium chloride, 3 parts dichlorozinc, 2 parts CBD, 2 parts pentitol and 1 part 2-(2-hydroxyethylamino)ethanol; 1 part ethane-1,2-diol, 3 parts ethylazanium chloride and 2 parts CBD; 5 parts choline chloride, 5 parts 1-tetradecanol and 5 parts CBD; 1 part choline chloride, 2 parts tetrabutylazanium chloride, 3 parts sorbose and 3 parts CBD; 1 part borneol, 1 part CBD, 1 part 1,3-propanediol and 4 parts stearic acid; and 1 part formic acid, 1 part CBD and 5 parts 1-tetradecanol; 5 parts CBD, 1 part menthol and 1 part lauric acid; 2 parts CBD, 1 part menthol; 3 parts CBD, 2 parts menthol and 1 part lauric acid; 3 parts CBD, 1 part menthol and 1 part glycol; and 3 parts CBD, 1 part undecanoic acid and 1 part eucalyptol.

30. The eutectic mixture of claim 1 further comprising menthol.

31. A pharmaceutical composition comprising:
a eutectic mixture comprising:
a cannabinoid and a least one compound selected from the group consisting of an alkyl amine, an alcohol and a carboxylic acid;
wherein said eutectic mixture has a melting point low enough to remain liquid at ambient temperature.

32. The pharmaceutical composition of claim 31 wherein said cannabinoid is defined by General Formula: 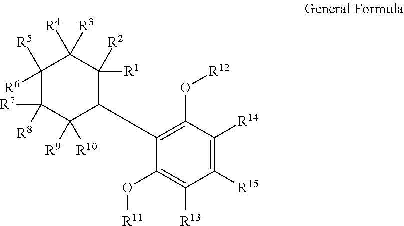
wherein:
R¹-R¹⁰ are independently selected from H or an alkyl of 1-10 carbons; alkenyl of up to 10 carbons; or groups on adjacent carbons may be taken together to form an alkene;
R¹¹ and R¹² are independently H or R¹¹ and R¹⁰ may be taken together to represent —C(R¹⁹)₂—;
R¹³-R¹⁵ independently represent H or an alkyl of 1-8 carbons; and
each R¹⁹ independently represents H or an alkyl of 1-5 carbons.

33. The pharmaceutical composition of claim 32 wherein each of R¹, R⁵, R⁶, R⁷, R⁸ and R⁹ is H.

34. The pharmaceutical composition of claim 32 wherein R² and R³ are taken together to form an alkene.

35. The pharmaceutical composition of claim 32 wherein R⁴ is —CH₃.

36. The pharmaceutical composition of claim 32 wherein R¹⁰ is —C(═CH₂)CH₃;

37. The pharmaceutical composition of claim 32 wherein R¹³ and R¹⁴ are H.

38. The pharmaceutical composition of claim 32 wherein R¹⁵ is an alkyl of 1-8 carbons.

39. The pharmaceutical composition of claim 38 wherein R¹⁵ is —(CH₂)₄CH₃.

40. The pharmaceutical composition of claim 32 wherein each R¹⁹ is —CH₃.

41. The pharmaceutical composition of claim 32 wherein said cannabinoid is defined by Formula I: 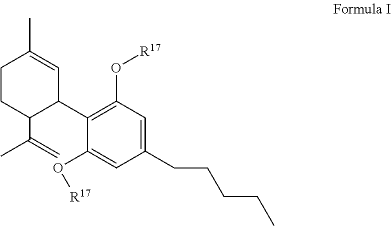
wherein each R¹⁷ is independently selected from H, C(O)C₆H₄OH, —C(O)CH═CHC₆H₅, —C(O)(CH₂)₇CH₃, or —C(O)(CH₂)₉CH₃.

42. The pharmaceutical composition of claim 41 wherein both R¹⁷ groups are the same.

43. The pharmaceutical composition of claim 42 wherein both R¹⁷ groups are hydrogen.

44. The pharmaceutical composition of claim 32 wherein said cannabinoid is defined by Formula II: 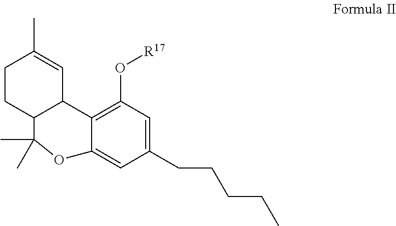
wherein R¹⁷ is independently selected from H, C(O)C₆H₄OH, —C(O)CH═CHC₆H₅, —C(O)(CH₂)₇CH₃, or —C(O)(CH₂)₉CH₃.

45. The pharmaceutical composition of claim 44 wherein R¹⁷ is hydrogen.

46. The pharmaceutical composition of claim 31 wherein said cannabinoid is selected from the group consisting of cannabidiol, tetrahydrocannabinol, cannabichromene, cannabichromevarin, cannabidiphorol, cannabidivarinic acid, cannabielsoin, cannabigerolic acid, cannabigerolic acid monomethyl ether, cannabigerovarinic acid, cannabicyclolic acid, cannabinolic acid, cannabicitranic acid, cannabivarinic acid, tetrahydrocannabinolic acid, tetrahydrocannabiorcolic acid, tetrahydrocannabiphorolic acid, tetrahydrocannabinolic acid, tetrahydrocannabivarinic acid, cannadibiphorol, cannabidivarin, cannabielsoin, cannabigerol, cannabigerol monomethylether, cannabigerovarin, cannabigerol monomethylether, cannabigerovarin, cannabicyclol, cannabinol, cannabicitran, cannabivarin, tetrahydrocannabiorcol, tetrahydrocannabiphorol and tetrahydrocannabivarin.

47. The pharmaceutical composition of claim 46 wherein said cannabinoid is selected from the group consisting of cannabidiol and tetrahydrocannabinol.

48. The pharmaceutical composition of claim 47 wherein said eutectic mixture further comprises menthol.

49. The pharmaceutical composition of claim 47 wherein said cannabinoid is cannabidiol tetrahydrocannabinol.

50. The pharmaceutical composition of claim 31 wherein said eutectic mixture comprises a cannabinoid and an alkyl amine.

51. The pharmaceutical composition of claim 50 wherein alkyl amine is an alkynol amine.

52. The pharmaceutical composition of claim 50 wherein alkyl amine is selected from the group consisting of alkyl azanium halides, choline halide, ethyl-(2-hydroxyethyl)-dimethylazanium chloride, diethyl-(2-hydroxyethyl)-azanium chloride, benzyl(triethyl)azanium chloride, benzyl(trimethyl)azanium chloride and tetrabutylazanium chloride; 2-[bis(2-hydroxyethyl)amino]ethanol, 2-(2-hydroxyethylamino)ethanol, 2-aminoethanol, tetraethylamine chloride, 1,3 dimethyl urea, 1,1-dimethyl urea, trimethyl glycine (betaine), carnitine, ethyl(2-hydroxyethyl)dimethylammonium chloride, diethanolamine and ethanol amine.

53. The pharmaceutical composition of claim 52 wherein azanium halides is selected from the group consisting of ethylazanium chloride, choline chloride, choline fluoride, ethyl-(2-hydroxyethyl)-dimethylazanium chloride, benzyl(triethyl)azanium chloride and tetrabutylazanium chloride.

54. The pharmaceutical composition of claim 31 wherein said eutectic mixture comprises a cannabinoid and an alcohol.

55. The pharmaceutical composition of claim 54 wherein said alcohol is selected from a mono-alcohols and a polyol.

56. The pharmaceutical composition of claim 55 wherein said alcohol is selected from the group consisting of methanol, propane-1-ol, hexafluoroisopropanolmenthol, tetradecane-1-ol, 1-tetradecanol, borneol, vanillin, ethylene glycol, glycerol, ethane-1,2-diol, butane-1,2-diol, butane-1,3-diol, 1,4 butanediol, butane-1,2,3,4-tetrol, 1,3-propanediol, 1,6-hexanediol, pentinol, hexitol, ribose, glucose, galactose, lactose, maltose and alpha-D-glucopyranoside.

57. The pharmaceutical composition of claim 31 wherein said eutectic mixture comprises a cannabinoid and a carboxylic acid.

58. The pharmaceutical composition of claim 57 wherein said carboxylic acid is selected from the group consisting of tetradecanoic acid, 2-aminopentanedioic acid, lauric acid, nonanoic acid, formic acid, dodecanoic acid, adipic acid, stearic acid, phthalic acid, undec-10-enoic acid, undecanoic acid, 1,3,4,5,6-pentahydroxyhexan-2-one, glutamic acid, octanoic acid, palmitic acid, hexanoic acid and trimesic acid.

59. The pharmaceutical composition of claim 31 wherein said eutectic mixture is selected from the group consisting of: 0.9-1.1 part ethylazanium chloride, 0.9-1.1 part tetradecanoic acid and 4.5-5.5 parts CBD; 0.9-1.1 part propan-1-ol, 2.7-3.3 parts choline chloride and 0.9-1.1 part CBD; 0.9-1.1 part choline chloride, 0.9-1.1 part ethyl-(2-hydroxyethyl)-dimethylazanium chloride, 2.7-3.3 parts menthol and 4.5-5.5 parts CBD; 0.9-1.1 part choline chloride, 2.7-3.3 parts 2-aminopentanedioic acid, 2.7-3.3 parts CBD and 1.8-2.2 parts ethylazanium chloride; 0.9-1.1 part 2-[bis(2-hydroxyethyl)amino]ethanol, 0.9-1.1 part water and 4.5-5.5 parts CBD; 0.9-1.1 part 2-(2-hydroxyethylamino)ethanol, 0.9-1.1 part tetradecan-1-ol and 4.5-5.5 parts CBD; 0.9-1.1 part choline chloride, 0.9-1.1 part undec-10-enoic acid, 0.9-1.1 part 2-[bis(2-hydroxyethyl)amino]ethanol and 4.5-5.5 parts CBD; 1.8-2.2 parts glycerol, 0.9-1.1 part dichlorozinc and 0.9-1.1 part CBD; 0.9-1.1 part choline chloride, 3.6-4.4 parts CBD and 4.5-5.5 parts urea; 2.7-3.3 parts butane-1,2-diol, 0.9-1.1 part ethylene glycol and 0.9-1.1 part CBD; 1.8-2.2 parts choline chloride, 2.7-3.3 parts benzyl(triethyl)azanium chloride, 0.9-1.1 part CBD, 0.9-1.1 part 1,6-hexanediol and 0.9-1.1 part lauric acid; 0.9-1.1 part (CH₃CH₂)₄N⁺Cl⁻, 0.9-1.1 part propan-1-ol, 0.9-1.1 part CBD and 0.9-1.1 part CH₃(CH₂)₇COOH; 0.9-1.1 part choline chloride, 2.7-3.3 parts CBD and 4.5-5.5 parts 1,3-dimethylurea; 0.9-1.1 part choline chloride, 1.8-2.2 parts CBD, 2.7-3.3 parts formic acid and 4.5-5.5 parts betaine; 0.9-1.1 part choline chloride, 3.6-4.4 parts ethanolamine, 0.9-1.1 part CBD and 1.8-2.2 parts glycerine; 0.9-1.1 part choline chloride, 4.5-5.5 parts dodecanoic acid, 3.6-4.4 parts adipic acid and 0.9-1.1 part CBD; 0.9-1.1 part ethyl-(2-hydroxyethyl)-dimethylazanium chloride, 1.8-2.2 parts CBD and 1.8-2.2 parts ethylazanium chloride; 1.8-2.2 parts ethanolamine, 0.9-1.1 part propan-1-ol and 1.8-2.2 parts CBD; 1.8-2.2 parts 1,4 butanediol, 1.8-2.2 parts 1,3-propanediol and 3.6-4.4 parts CBD; 0.9-1.1 part ethyl-(2-hydroxyethyl)-dimethylazanium chloride, 2.7-3.3 parts dichlorozinc, 1.8-2.2 parts CBD, 1.8-2.2 parts pentitol and 0.9-1.1 part 2-(2-hydroxyethylamino)ethanol; 0.9-1.1 part ethane-1,2-diol, 2.7-3.3 parts ethylazanium chloride and 1.8-2.2 parts CBD; 4.5-5.5 parts choline chloride, 4.5-5.5 parts 1-tetradecanol and 4.5-5.5 parts CBD; 0.9-1.1 part choline chloride, 1.8-2.2 parts tetrabutylazanium chloride, 2.7-3.3 parts sorbose and 2.7-3.3 parts CBD; 0.9-1.1 part borneol, 0.9-1.1 part CBD, 0.9-1.1 part 1,3-propanediol and 3.6-4.4 parts stearic acid; 0.9-1.1 part formic acid, 0.9-1.1 part CBD and 4.5-5.5 parts 1-tetradecanol; 4.5-5.5 parts CBD, 0.9-1.1 part menthol and 0.9-1.1 part lauric acid; 1.8-2.2 parts CBD, 0.9-1.1 part menthol; 2.7-3.3 parts CBD, 1.8-2.2 parts menthol and 0.9-1.1 part lauric acid; 2.7-3.3 parts CBD, 0.9-1.1 part menthol and 0.9-1.1 part glycol; 2.7-3.3 parts CBD, 0.9-1.1 part undecanoic acid and 0.9-1.1 part eucalyptol.

60. The pharmaceutical composition of claim 59 wherein said eutectic mixture is selected from the group consisting of: selected from the group consisting of: 1 part ethylazanium chloride, 1 part tetradecanoic acid and 5 parts CBD; 1 part propan-1-ol, 3 parts choline chloride and 1 part CBD; 1 part choline chloride, 1 part ethyl-(2-hydroxyethyl)-dimethylazanium chloride, 3 parts menthol and 5 parts CBD; 1 part choline chloride, 3 parts 2-aminopentanedioic acid, 3 parts CBD and 2 parts ethylazanium chloride; 1 part 2-[bis(2-hydroxyethyl)amino]ethanol, 1 part water and 5 parts CBD; 1 part 2-(2-hydroxyethylamino)ethanol, 1 part tetradecan-1-ol and 5 parts CBD; 1 part choline chloride, 1 part undec-10-enoic acid, 1 part 2-[bis(2-hydroxyethyl)amino]ethanol and 5 parts CBD; 2 parts glycerol, 1 part dichlorozinc and 1 part CBD; 1 part choline chloride, 4 parts CBD and 5 parts urea; 3 parts butane-1,2-diol, 1 part ethylene glycol and 1 part CBD; 2 parts choline chloride, 3 parts benzyl(triethyl)azanium chloride, 1 part CBD, 1 part 1,6-hexanediol and 1 part lauric acid; 1 part (CH₃CH₂)₄N⁺Cl⁻, 1 part propan-1-ol, 1 part CBD and 1 part CH₃(CH₂)₇COOH; 1 part choline chloride, 3 parts CBD and 5 parts 1,3-dimethylurea; 1 part choline chloride, 2 parts CBD, 3 parts formic acid and 5 parts betaine; 1 part choline chloride, 4 parts ethanolamine, 1 part CBD and 2 parts glycerine; 1 part choline chloride, 5 parts dodecanoic acid, 4 parts adipic acid and 1 part CBD; 1 part ethyl-(2-hydroxyethyl)-dimethylazanium chloride, 2 parts CBD and 2 parts ethylazanium chloride; 2 parts ethanolamine, 1 part propan-1-ol and 2 parts CBD; 2 parts 1,4 butanediol, 2 parts 1,3-propanediol and 4 parts CBD; 1 part ethyl-(2-hydroxyethyl)-dimethylazanium chloride, 3 parts dichlorozinc, 2 parts CBD, 2 parts pentitol and 1 part 2-(2-hydroxyethylamino)ethanol; 1 part ethane-1,2-diol, 3 parts ethylazanium chloride and 2 parts CBD; 5 parts choline chloride, 5 parts 1-tetradecanol and 5 parts CBD; 1 part choline chloride, 2 parts tetrabutylazanium chloride, 3 parts sorbose and 3 parts CBD; 1 part borneol, 1 part CBD, 1 part 1,3-propanediol and 4 parts stearic acid; and 1 part formic acid, 1 part CBD and 5 parts 1-tetradecanol; 5 parts CBD, 1 part menthol and 1 part lauric acid; 2 parts CBD, 1 part menthol; 3 parts CBD, 2 parts menthol and 1 part lauric acid; 3 parts CBD, 1 part menthol and 1 part glycol; and 3 parts CBD, 1 part undecanoic acid and 1 part eucalyptol.

61. The pharmaceutical composition of claim 31 further comprising menthol.

62. The pharmaceutical composition of claim 31 further comprising at least one of a carrier and a pharmaceutically active ingredient.

63. The pharmaceutical composition of claim 62 wherein said carrier is an oil.

64. The pharmaceutical composition of claim 62 wherein said pharmaceutically active ingredient is selected from the group consisting of a cough suppressant, an aromatic and an analgesic.

65. The pharmaceutical composition of claim 62 further comprising eucalyptol or eucalyptus oil.
